(19) United States
(12) Patent Application Publication
(10) Pub. No.: US 2023/0110571 A1
(43) Pub. Date: Apr. 13, 2023

(54) LED and AUDIO FRAGRANT OIL DIFFUSER

(71) Applicant: Andrew Walker, JR., Culver City, CA (US)

(72) Inventor: Andrew Walker, JR., Culver City, CA (US)

(21) Appl. No.: 17/496,622

(22) Filed: Oct. 7, 2021

(51) Int. Cl. A61L 9/03; F21V 29/503; F21V 29/60; F21V 33/00; F21V 23/00; H04R 1/02; A61L 9/01

(52) U.S. Cl. CPC A61L 9/032; F21V 29/503; F21V 29/60; F21V 33/004; F21V 23/003; H04R 1/028; A61L 9/01; F21Y 2115/10

(57) ABSTRACT

A warmer including base, top cover, warming devices for scented oil. Device includes fan, speaker, heating rod, LED bulb, wi-fi capabilities, control device remotely. The warmer is in a heat conductive relationship with the cover so that scented oil in the open channel is warmed by the warming device. Warmer's lighting system, including: a substrate; first set of light emitting elements and second set of light emitting elements configured to emit visible light having fixed first and second color parameter; diffuser cooperatively enclosing first and second sets of light emitting elements with substrate; communication module including antenna; processor connected to communication module, first set of light emitting elements, and second set of light emitting elements, processor configured to independently control relative intensities of first and second set of light elements to cooperatively emit light having target color parameter value, wherein target color parameter value is received from communication module.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever

### BACKGROUND

### Field of the Invention

Embodiments disclosed herein generally relate to warmers for scented oils and wax fragrances. More particularly, the invention relates to a new and useful low manufacturing cost, dynamically adjustable fragrant warmer LED lighting system in the lighting systems and warmer field.

### Description of the Related Art

The release of scents to cover or mask unpleasant aromas and/or to provide a desired aroma have been practiced for many years. The heating of scented oils is one known method of releasing scents.

By looking at prior art multiple types of advancements have been seen in similar regards. For instance, a Warmers for scented oils bearing US patent 2,011,0286726A1 is issued to Partylite Worldwide Inc. The patent discusses a warmer for scented oil is provided. The warmer includes a base, a cover removably positioned on the base, and a warming device. The cover has a substantially spherical shape with an opening at an upper apex and an open channel formed in the cover at the upper apex around an outer periphery of the opening. The open channel is configured to receive the scented oil. The warming device is in a heat conductive relationship with the cover so that scented oil in the open channel is warmed by the warming device. The warming device can be one or more of a lighting device with an incandescent light bulb, a resistance heater with or without a lighting device such as a light emitting diode, or a candle.

Another patent on Candle-powered aroma generator bearing U.S. Pat. No. 4,781,895A is issued to Donald Spector. The patent discusses a candle-powered aroma generator in a lantern format housing a candle holder within a cylindrical container provided with a translucent shell mounted on a base and having a removable cover thereover whose roof has a central well. Below the bottom of the well is the burning wick of a candle supported in the holder, the candle flame heating the bottom which then functions as a miniature hot plate. Received in the well and resting on the hot plate is a replaceable aroma cartridge within whose cavity is a porous pad that partitions the cavity into upper and lower air chambers. The lower chamber is in heat transfer relation to the hot plate and the upper chamber has a vent therein. The pad is impregnated with a volatile liquid fragrance, and the heated air in the lower chamber produces a positive pressure therein which forces the air through the pad to volatilize the fragrance. The resultant vapor is discharged into the atmosphere through the vent.

Another patent on Scent-generating lamp using mating parts bearing U.S. Pat. No. 4,965,490A is issued to Osram Sylvania Inc. The patent relates to a vapor-generating electric lamp includes a sealed lamp envelop having a concave depression therein, an incandescent filament mounted within the lamp envelope, a lamp base, and a replaceable, solid insert in the concave depression. The insert produces a scent when it is warmed by heat from the filament.

Preferably, the insert is a polyamide impregnated with a scent and includes a portion that matches the shape of the depression in the lamp envelope. The depression for holding the insert is preferably located at the opposite end of the lamp from the base. When the lamp is used in a base-down orientation, the insert is held in the depression by gravity.

Another patent on Aroma dispenser for candle bearing U.S. Pat. No. 6,328,935B1 is issued to Custom Essence Inc. The patent relates to an aroma dispenser comprises a flame source for providing heat, an open, porous annular ceramic substrate adapted for absorbing and reversibly retaining a quantity of at least one aromatic fluid therein, a support structure for holding the porous substrate in a coaxial spaced-apart relationship above the flame source, whereby heat generated by the flame source induces the retained aromatic fluid within the substrate to disperse into the ambient air.

A patent on warmers for scented oils bearing US patent 2,011,0286726A1 is issued to Partylite Worldwide Inc. The patent relates to a warmer for scented oil is provided. The warmer includes a base, a cover removably positioned on the base, and a warming device. The cover has a substantially spherical shape with an opening at an upper apex and an open channel formed in the cover at the upper apex around an outer periphery of the opening. The open channel is configured to receive the scented oil. The warming device is in a heat conductive relationship with the cover so that scented oil in the open channel is warmed by the warming device. The warming device can be one or more of a lighting device with an incandescent light bulb, a resistance heater with or without a lighting device such as a light emitting diode, or a candle.

Another Canadian patent 2,449,734A1 on Candle warmer is issued to Pende Inc. The patent relates to a scent dispersion system (10) including a heating device (14) having a top surface, and a container (16) of scented wax disposed upon the top surface of the heating device (14). Upon heating, scent from the wax is caused to disperse into the surrounding environment. The system (10) may further include an air circulating fan (34) associated with the heating device (14), for circulating air around the container (16) of scented wax, to help disperse the scent.

There are multiple inventions that have been proposed art regarding brining an advancement and enhanced utility regarding warmers for scented oils and wax fragrances. However, it is having been determined by the present disclosure that there is a need for warmers for scented oils that overcome, alleviate, and/or mitigate one or more deleterious effects of prior art warmers.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

It is a general object of the present invention to provide an improved composition of warmer for scented oil and wax.

The includes a base, a cover removable positioned on the top, and warming devices. The cover has a substantially curvy shape with a crown cone shape top piece that sits inside the aperture of the device with an opening at an upper apex and an open channel formed in for the aluminum alloy container that has an open channel is configured to receive the scented oil or wax. The warming device is a heat conductive relationship with the aluminum alloy container so that scented oil or wax in the open channel container is warmed by the warming device. The warning device can be one a resistance heater rod or lighting devices with a LED smart light bulb, developed with polychrome technology, that give the user with wi-fi capabilities to turn the device on and off, select the color of the smart light bulb, temperature and control the device remotely. Also, the device has an audio equipped fan is disclosed as having a housing, and a grille connected to the housing, and defining first openings through which air may flow while the fan is rotated, and second openings through which sound may travel. The fan also has an annular lighting array configured to selectively provide lighting in a high power or a low power configuration, and speakers connected to the grille and aligned on the interior side of the grille with the second openings of the grille so that sound may travel through the grille.

A lighting system, including: a substrate defining a first broad face; a first set of light emitting elements configured to emit visible light having a fixed first color parameter; a second set of light emitting elements configured to emit visible light having a fixed second color parameter different from the first color parameter; a diffuser cooperatively enclosing the first and second sets of light emitting elements with the substrate; a communication module including an antenna; and a processor operatively connected to the communication module, the first set of light emitting elements, and the second set of light emitting elements, the processor configured to independently control relative intensities of the first and second set of light emitting elements to cooperatively emit light having a target color parameter value, wherein the target color parameter value is received from the communication module.

The warming device can be, in some embodiments, a lighting device with a smart LED light bulb with polychrome technology. The lighting device can be secured to a base of the enclosure so that the enclosure shields substantially all of the led smart light bulb. Here, the enclosure can include a decorative pattern defined on an external surface and/or one or more portions of the enclosure can be at least partially translucent. The warming device can also be, in other embodiments, resistance heater rod. Resistance heater rod can be secured to a base of the enclosure. These embodiments, the warmer can optionally include a lighting device such as, but not limited to, a light emitting diode. Again, the enclosure can include a decorative pattern defined on an external surface and/or one or more portions of the enclosure can be at least partially translucent. The warming device in still other embodiments can be a heating steam rod or led smart light bulb that is removably position able within the enclosure so that the enclosure shields substantially all of the heating rod or smart light bulb with the heating rod or smart light bulb being at a center of the opening.

The warmer can optionally include at least one vent opening defined in the enclosure. The vent opening can be, for example, a plurality of evenly spaced openings defined near the top of the enclosure for the introduction of the reservoir container and ventilation for the bulb. Also, the enclosure can include a decorative pattern defined on an external surface and/or one or more portions of the enclosure can be at least partially translucent. A warmer for scented oil or wax having a base, a cover, a vent opening defined between the crown and the base, and a heating rod and led smart light bulb is also provided. The device has an opening at an upper apex and is removably positioned on the top. An open channel is formed in the rim around an outer periphery of the opening, with the open channel container being configured to receive the scented oil therein. The heating rod or the led smart light bulb is removably position able on the base with the heating rod or the led smart light bulb being at a center of the opening. In this manner, the heating channel container is in a heat conductive relationship with the heating rod or the smart light bulb so that scented oil in the open channel container is warmed by ether the heating rod or the smart light bulb. A fan assembly supported on a ceiling, comprising: a housing having an air inlet, an air outlet, and an interior positioned between the inlet and outlet; a motor and a fan driven by the motor, the fan being supported in the interior of the housing and being operable to move air from a room; a grille connected to the housing and extending across the air inlet of the housing, having an interior side and an exterior side and defining a plurality of first openings through which air may flow while the fan is on and a plurality of second openings through which sound may travel in which the fan, smart bulb, speaker, and heating rod may be place in various ways attached to the base.

In various embodiments, the cover can include a decorative pattern defined on an external surface and/or the cover can be at least partially translucent.

Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments. reference USD593191s prior filing.

FIG. 1 is an exploded perspective view of a fragrant oil burning lamp, showing my new fragrant oil burning warmer and illustrating a top portion removable from a base portion and a fragrant oil reservoir tube that normally rests in the top rim portion according to an exemplary embodiment of the present disclosure;

FIG. 2 is a front perspective view of the warmer of FIG. 1 having portions of the cover cut away to show first and second embodiment of warming devices according to an exemplary embodiment of the present disclosure;

FIG. 3 is a rear elevational view of the invention;

FIG. 4 is a second side view of the warmer of FIG. 1, which is offset from the first side view of FIG. 3 by one hundred and eighty degrees;

FIG. 5 is a right-side elevational view of the invention;

FIG. 6 is a top plan view of the invention;

FIG. 7 is a bottom plan view of the invention; and,

FIG. 8 is a top plan view of the invention, illustrated with a top of the lamp removed to expose a fragrant oil reservoir, and

FIG. 9 is a front perspective view of the warmer of FIG. 1 having portions of the cover cut away to show two of the embodiments of a warming devices, fan, air vent, and speaker according to an exemplary embodiment of present disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION OF THE INVENTION

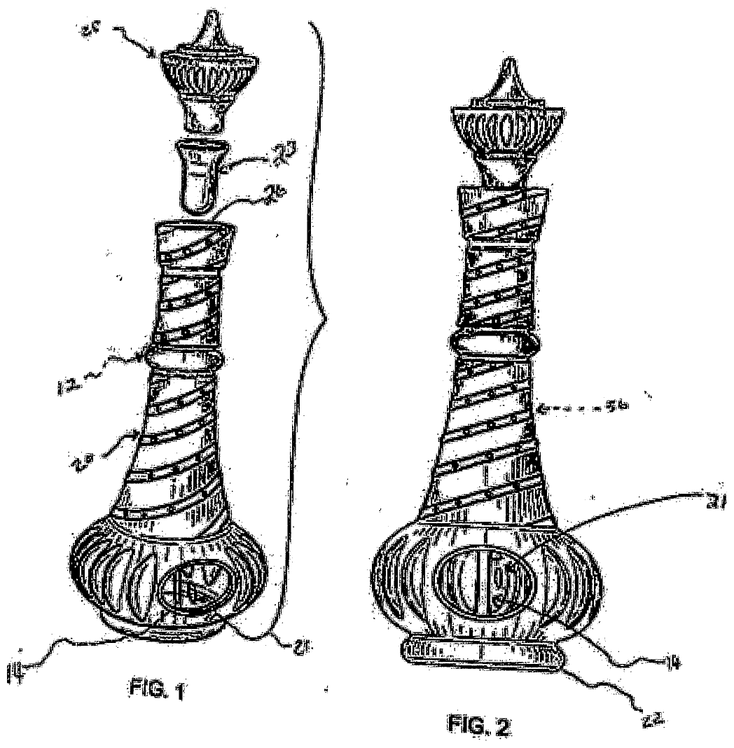

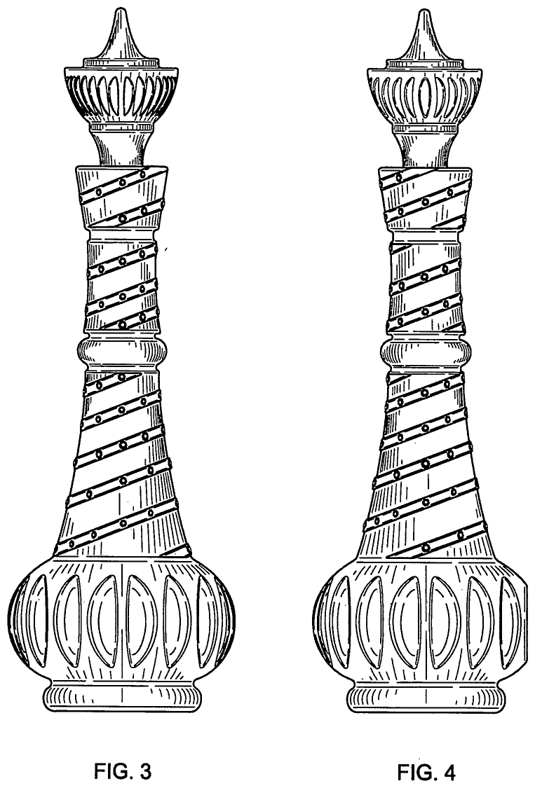

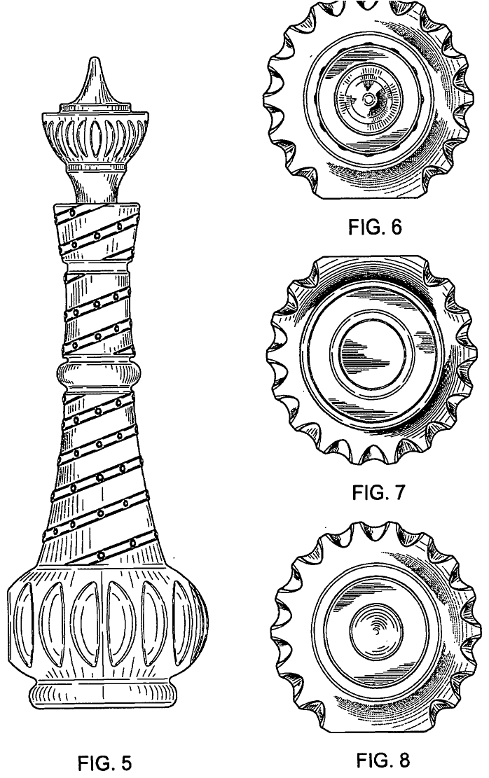

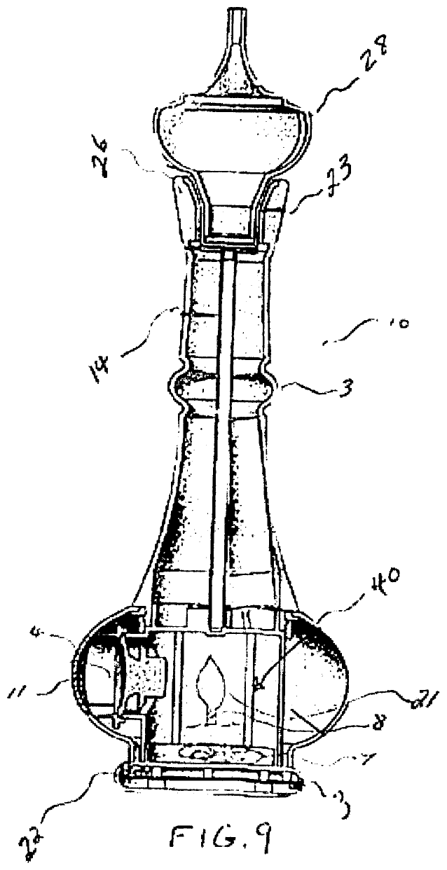

Referring to the drawings and in particular to FIGS. 1 through 9, an exemplary embodiment of a warmer for scented oils is shown and is generally referred to by reference numeral 12. Warmer 12 includes an enclosure 12 and a warming device 21 and 14. In the embodiment of FIG. 9, warming device 21 is illustrated by way of example as a LED smart light bulb 21, which is shown in phantom and the resistance heating rod 14 is illustrated by way of pipe connected to the fragrant oil reservoir container 23 and fan 7 for current control and speaker 4 wherein outputs audio sound from said warmer 12.

Advantageously, warmer 12 is configured to use the heat generated by warming device 21 or the heating rod 14 to heat enclosure 12. Enclosure 12 conducts the heat received from warming device 21 or 14 to warm scented oil or wax (not shown) within an open container reservoir 23 defined in the enclosure, where the open container reservoir is configured to receive scented oil or wax therein, in the embodiment of FIG. 9. As used herein, the term “scented oil” shall mean any composition, in a liquid form, a semi-solid form, or a solid form, essential oils, that includes a scent within the composition, whereas the scent is released upon heating of the composition to a desired temperature at a known release rate.

As used herein, the term “scented wax” or oil referring to ether fragrant oil or essential oil shall mean any composition, in a liquid form, a semi-solid form, or a solid form, that includes a scent within the composition, whereas the scent is released upon heating of the composition to a desired temperature at a known release rate.

In some embodiments, warmer 12 includes open container oil or wax reservoir 23 pre-filled with scented oil or wax in a solid or semi-solid form. In other embodiments, warmer 12 includes an empty open container oil or wax reservoir 23, which can be filled at the time of use by the user.

In the illustrated embodiment, enclosure 12 includes three components, namely, crown cover 28 a base 22 and a container oil and wax reservoir 23. Of course, it is contemplated by the present disclosure for enclosure 12 to be a single unitary component or for the enclosure to include more than three components, which fit together to form the desired enclosure around the warming device 12 wherein said least one vent 3 opening comprises a plurality of evenly spaced indentations defined in said cover 12.

Cover crown 28 includes an inner circular opening or surface with an opening that sit inside of the oil container 23 in which both sits inside the upper aperture of 12 and as 26 defined therethrough. Open container oil or wax reservoir 23 is formed in cover 28 at upper rim of warmer 12 around an inner aperture of opening 26. Opening 26 allows light and heat generated by the LED smart light bulb 21 or rod 14 to escape cover 12. In addition, cover 12 is in a heat conductive relationship with warming device 21 or rod 14 so that the scented oil or wax in open container oil or wax reservoir 23 is warmed by ether warming device 21/14. Cover 12 removably positioned on base 22 so that the user can selectively access LED smart light bulb 21 or rod 14 as needed for clean, and/or replacement of the LED smart light bulb 21, 23 and an outer upstanding rim that rest on the rim of the inner rim of 12. In this manner, scented oil or wax within the open container oil and wax reservoir 23 is retained by rims 12, 26 so that the scented oil and wax does not flow down an outer surface 12 of cover or flow into the inner area of the cover 12 through opening 26. Crown cover 28 includes an inner upstanding rim on the base of the crown cover 28 around the inner of opening 26. In this manner, the crown cover 28 and the oil reservoir container 23 can sit firmly into the top inner openings of the cover 12.

Led smart light Bulb 21 and the rod 14 is repositioned on base 22 so that cover 12 shields or encloses substantially all of the smart bulb 21, rod 14 and so that the smart bulb 21 and rod 14 is at a center of opening 26. In this manner, warmer 12 allows light from the smart light bulb 21 or heat from the rod 14 to exit through the top of the cover 12 of the container reservoir 23, yet still provides sufficient heat to release the scent from the scented oil or wax located inside of the container reservoir 23. Also, the crown cover 28 has apertures located within the cover of it, this way, the user can choose to keep the crown cover 28 on the device 12 while in use or have the option to take the crown cover 28 off and release the scent.

In other embodiments, cover 12 can include one or more decorative patterns 36 (only one shown) defined on external surface 12. Device can be powered on and off with the push button 33 on the base of the device 12 or with wi-fi through the smart bulb 21. Also, it is contemplated by the present disclosure for cover 12 to be at least partially translucent to allow at least a portion of the light generated by smart light bulb 21 to escape the cover. As used herein, the term “translucent” shall mean that at least some portion of visible light can pass through the cover by way of openings in the cover and/or by way of the property of the materials from which the cover is made allowing light to pass therethrough.

Enclosure 12 can be formed of any material conduct heat from warming device 21 or rod 14 to open container reservoir 23 and provide, when desired, the aforementioned translucent property and LED lighting desired by the LED smart bulb in a preferred embodiment, enclosure 12 is formed of a material selected from the group consisting of glass, ceramic, plastic, and any combinations thereof. In the illustrated embodiments, cover 12 is shown having a substantially curvy shape. Of course, it is contemplated by the present disclosure for cover 12 to have any desired shape.

It should also be recognized that warmer 12 is described above using LED smart light bulb 21 and the resistance heating rod 14 as the warming devices 21/14. Of course, is contemplated by the present disclosure for warming devices 21/14 to include any device configured to provide heat to enclosure 12 in sufficient amount so as to release the scent from the scented oil or wax in the open channel container reservoir 23 of the enclosure. Referring now to FIG. 9, warmer 12 is illustrated using a LED smart light bulb 21 as warming device 21. Here, smart LED light bulb 21 and the rod 14 is formed as part of a lighting device 40, which is secured to base 22. Cover 12 shields substantially all of rod 14 and LED smart light bulb 21. As discussed in detail above, cover 12 can include decorative pattern 36 defined on an external surface 12 thereof and/or the cover can be at least partially translucent. Lighting/heating device 21 and heating rod 14 can be selectively placed in electrical communication with a power source (not shown) such as, but not limited to, a battery or an electrical outlet. In some said base 22 Vent openings 3 defined between cover 12 and said base 22. Fan 7 and vent opening 3 can ensure that the LED smart bulb 21 has sufficient oxygen and cool air to operate properly and provides a convective air current through the warmer 12 enclosed in a separate compartment is fan 7, and LED smart bulb 21 at the base 22 of the warmer 12.

The lighting system 21, includes multiple sets of electromagnetic signal emitting elements and a processor configured to control operation of the multiple sets of EM signal emitting elements. The lighting system 21, functions to emit electromagnetic signals, such as light, having at least one, more preferably at least two, adjustable properties, wherein the adjustable properties can be color temperature, wavelength, intensity, or any other suitable electromagnetic property. The lighting system 21, is preferably a light bulb 21, (as shown in FIG. 9), but can alternatively be incorporated into any other suitable component or utilized in any other suitable application. The lighting system 21, confers several benefits over conventional lighting systems. First, by using multiple sets of light emitting elements that have substantially fixed emission properties that are substantially cheaper than light emitting elements having variable emission properties, the lighting system 21, enables dynamic adjustment of the properties of the resultant light emitted by the lighting system 21, as a whole. Second, by incorporating sets of light emitting elements having emission properties outside of the human-visible spectrum (e.g., outside of approximately 390 to 700 nm), the lighting system 21, can enable higher-resolution imaging at the respective wavelengths. For example, incorporating a set of infrared emitting elements into the lighting system 21, can enable better IR imaging resolution for security, low-light monitoring, or thermo-monitoring applications. Third, by incorporating EM signal emitting elements having at least one or more variable parameters, the lighting system 21, enables dynamic aesthetic adjustment to substantially match or accommodate for the EM signal quality (e.g., light quality) emitted by previously installed systems in the space.

The electromagnetic signal emitting element (EM signal emitting element) of the lighting system 21, is configured to emit electromagnetic signals having a set of properties. The EM signal emitting element (or combination thereof can function to illuminate a physical area with light having a specified set of properties. The EM signal emitting element (or combination thereof) can additionally or alternatively, function communicate data to other systems (e.g., devices, the warmer 12, other lighting system) within a communication range. However, the EM signal emitting element can perform any other suitable functionality. The EM signal emitting element can include an active surface configured to emit the EM signal, but can alternatively emit the signal in any other suitable manner. The EM signal emitting element is preferably mounted to the substrate more preferably a broad face of the substrate, but can alternatively, be mounted to the sides of the substrate, the diffuser, or to any other suitable lighting system component.

The EM signal emitting element preferably has fixed EM signal properties values, but can alternatively have variable EM signal property values. Alternatively, a limited subset of EM signal properties can have variable values, while the remaining EM signal properties of the set can have fixed values. For example, pulse frequency-independent properties, pulse width-independent properties, current-independent properties, voltage independent properties, or any other suitable subset of the property set can have fixed and/or variable values. When the electromagnetic property has a fixed value, the value is preferably fixed within a margin of error (e.g., 5% variation, manufacturing variation, etc.) of an original value, manufacturing value, specification value, or any other suitable value. The electromagnetic parameters are preferably light parameters, but can alternatively be thermal parameters, audio parameters, or any other suitable parameter. The light parameters can be light properties (e.g., wavelength, propagation direction, intensity, and frequency), color parameters (e.g., hue, saturation, color temperature, etc.), or include any other light parameter. However, any other suitable parameter can be fixed or varied.

For example, the EM signal emitting element can have a fixed wavelength and a variable intensity (e.g., wherein the element is dimmable, wherein the intensity is a current-dependent property). In a specific example, the EM signal emitting element (e.g., light emitting element) can only emit visible light having a fixed color temperature. Alternatively, the EM signal emitting element can only emit an invisible signal (e.g., IR light, RF signal). However, the EM signal emitting element can emit one or more wavelengths of light (concurrently or individually) or have any other suitable set of capabilities.

The EM signal emitting element can emit light (e.g., visible light, invisible light, such as IR, UV, etc.), RF, microwave, or any other suitable electromagnetic signal. Alternatively, or additionally, the lighting system 21, can include a sound or pressure wave emitter configured to emit a sound or pressure wave signal, or include any other suitable emitter. The sound or pressure wave signal can be an ultrasound signal, infrasound signal, or any other suitable sound or pressure wave signal. The EM signal emitting elements (e.g., light emitting elements) are preferably light emitting diodes (LEDs), but can alternatively be organic light emitting diodes (OLEDs), incandescent light bulbs, resistors, or any other suitable element configured to emit radiation. The light emitting elements can be visible light emitting elements, invisible light emitting elements, or emit light having any suitable property. The light emitting elements can emit a single wavelength of light (e.g., be a white LED, red LED, green LED, blue LED, cyan LED, IR LED, etc.), emit multiple wavelengths of light (e.g., be an RGB LED, RGBW LED 3-4 channel, etc.), or emit any suitable number of wavelengths. The EM signal emitting elements within a set are preferably substantially similar, but can alternatively be different. The EM signal emitting elements in different sets are preferably substantially similar, but can alternatively be different.

The lighting system 21, preferably includes a plurality of EM signal emitting elements, but can alternatively include a single EM signal emitting element or any suitable number of EM signal emitting elements. The plurality of EM signal emitting elements is preferably divided into multiple sets of EM signal emitting elements (e.g., one set, two sets, three sets, any other suitable number of sets), but can alternatively be controlled as the plurality. Each set of EM signal emitting elements preferably includes multiple EM signal emitting elements, but can alternatively include a single EM signal emitting element. Every set of EM signal emitting element preferably has the same number of EM signal emitting elements, but can alternatively have different numbers of EM signal emitting elements.

For example, a first set of light emitting elements can be low lumen-output elements, while the second set of light emitting elements can be high lumen-output elements, wherein the first set includes more light emitting elements to match the lumen output of the second set of light emitting elements. However, any suitable number of lights emitting element having any other suitable property can be used.

Each set of EM signal emitting elements preferably emits EM signals having at least one property that is different from the remaining sets of EM signal emitting elements (e.g., different wavelength, frequency, propagation direction, etc.), but can alternatively have the same EM signal properties. All EM signal emitting elements within a set can have substantially the same EM signal properties (e.g., within manufacturing error), share one or more EM signal property values (e.g., the same wavelength, phase, etc.), have entirely different electromagnetic property values, or have any other suitable set of EM signal property values. The parameter values associated with the different EM signal emitting element sets are preferably separated by a threshold value difference (e.g., opposing sides of a color spectrum, ctc.), but can alternatively be differentiated in any other suitable manner. The multiple sets of EM signal emitting elements are preferably arranged in a pattern along a substrate of the lighting system 21, but can alternatively be randomly arranged. The EM signal emitting elements are preferably substantially evenly distributed across the substrate, but can alternatively be unevenly distributed, such that the substrate includes portions with high concentrations of EM signal emitting elements, and other portions with low concentrations of EM signal emitting elements. The EM signal emitting element sets can be substantially evenly distributed across the substrate, be unevenly distributed across the substrate, or be otherwise distributed across the substrate.

In a first variation, the EM signal emitting element sets are concentrically arranged, wherein different EM signal emitting element sets can be arranged at different radial positions. In a second variation, the EM signal emitting element sets are arcuately arranged, wherein different EM signal emitting element sets can be arranged in different arcuate sections. In a third variation, the EM signal emitting elements of the sets arc randomly distributed and can be isotopically or non-isotopically distributed over the substrate. In a fourth variation, different EM signal emitting element sets are arranged within different contiguous portions of the substrate wherein the contiguous portions preferably do not overlap, but can alternatively overlap. In a fifth variation, an EM signal emitting element from each of a plurality of EM signal emitting element sets is included in a group, wherein the lighting system 21, includes multiple groups and the groups are evenly distributed across the substrate (dashed elements optional).

In a sixth variation, one or more EM signal emitting element sets can be arranged in the central portion of the substrate (e.g., the central portion of the substrate mounting face), and different EM signal emitting element set(s) can be arranged along the perimeter of the substrate (e.g., evenly or unevenly distributed along the perimeter). However, the multiple sets of EM signal emitting elements can be otherwise arranged on the substrate.

The EM signal emitting elements of a set are preferably connected in parallel, but can alternatively be connected in series. Different sets of EM signal emitting elements are preferably connected in parallel to the power source by a set of switches, but can alternatively or additionally be connected to different power control circuits or connected in any other suitable manner.

Each EM signal emitting element of a set can be independently indexed and controlled, indexed and controlled together with the other EM signal emitting elements of the set, indexed and controlled together with a subset of the light emitting elements of the set, or controlled in any other suitable manner. Each set of EM signal emitting elements is preferably independently indexed and controlled, but can alternatively be controlled with another set of EM signal emitting elements. The EM signal emitting elements of a subset can be EM signal emitting elements of the same set or EM signal emitting elements of different sets.

The EM signal emitting elements of the subset can be related by physical arrangement on the substrate (e.g., EM signal emitting elements aligned along a vector, such as a radial vector, longitudinal vector, lateral vector, or other vector; EM signal emitting elements arranged within a section of the substrate, such as an arcuate section, etc.), be otherwise related, or be unrelated.

The EM signal emitting elements are preferably indexed during or after manufacturing, but can alternatively be indexed in response to installation (e.g., into an appliance, a light fixture, or other power-connected component) or at any other suitable time. The index is preferably used to identify the EM signal emitting element, but can alternatively be used to determine parameters about the EM signal emitting element, or be used in any other suitable manner. For example, the index can be used to determine the EM signal emitting element location relative to a reference point. The reference point is preferably a lighting system 21, reference point on the lighting system 21, (e.g., an EM signal emitting element, a center point, etc.), wherein the location of the EM signal emitting element relative to the lighting system 21, reference point can be predetermined by the manufacturer or otherwise known. The position of the lighting system 21, reference point relative to an external reference point can be determined and used to select the EM signal emitting elements that should be selectively powered. Alternatively, the reference point can be an external reference point, such as a point in a room, a geographic location, compass direction, or any other suitable external reference point.

In one example, the lighting system 21, can include a first set of light emitting elements configured to emit light having a first color temperature (e.g., above 5,000K or any other suitable color temperature), and a second set of light emitting elements configured to emit light having a second color temperature (e.g., below 5,000K, between 2,700-3,000K, or any other suitable color temperature). However, the light emitting elements can be configured to emit light having any other suitable color temperature. Each of the above temperatures are at the set temperatures to warm the oil reservoir container 23, and cause the heated fragrant oil or wax inside of the container to be volatile into the air and release the scent at the desired point of the user from the warmer 12.

In a second example, the lighting system 21, can include a first set of light emitting elements configured to emit light at a first wavelength and a second set of light emitting elements configured to emit light at a second wavelength. In one variation, the first and second wavelengths are both within the visible spectrum (e.g., red and blue, respectively). In another variation, the first wavelength is in the visible spectrum and the second wavelength are outside of the visible spectrum (e.g., IR, UV, etc.). However, the light emitting elements can be configured to emit light at any another suitable wavelength. Also giving the user the desired visible spectrum from the warmer 12.

In a third example, the lighting system 21, can include: a first set of light emitting elements configured to emit visible light having a first fixed wavelength of visible light (e.g., white light having a fixed color temperature above 5,000 K or any other suitable color temperature); a second set of light emitting elements configured to emit visible light having a second fixed wavelength of visible light (e.g., white light having a fixed color temperature below 5,000K, between 2,700-3,000K, or any other suitable color temperature); and a third set of light emitting elements configured to emit a fixed wavelength of invisible light (e.g., IR light). The first, second, and third sets of light emitting elements can each be individually controlled (e.g., wherein the intensity of light emitted by the one set is independent from the intensity of light emitted by the other sets), or be controlled together (e.g., wherein the intensity of light emitted by the one set is dependent upon the intensity of light emitted by one or more of the other sets) while simultaneously warming the fragrant inside of the oil reservoir container 23 of the warmer 12. Each element or sub-group of the first, second, and/or third set can be independently indexed and controlled. Alternatively, all elements of a set are controlled together. However, the light emitting elements can be configured to emit light having any other suitable property and can be controlled in any suitable manner from the warmer 12.

The processor of the lighting system 21, functions to control EM signal emitting element operation based on lighting instructions received from a device. The processor can individually control the relative intensities of EM signals emitted by different EM signal emitting element sets (e.g., by controlling power provision to the multiple EM signal emitting element sets). In one variation, the processor can individually control a first and second set of light emitting elements to cooperatively emit light having a target color parameter value (e.g., wherein the light emitted by the first and second light emitting element are mixed by the diffuser to achieve the target light parameters). The processor can additionally, or alternatively receive control instructions for an external device (e.g., warmer 12), control an EM signal emitting element or set thereof to communicate the control instructions to a local external device, translate the control instructions from one communication protocol to another communication protocol, or perform any other suitable functionality. The processor is preferably electrically connected to every EM signal emitting element of the lighting system 21 but can alternatively be electrically connected to a subset of the EM signal emitting elements of a set; be electrically connected to some EM signal emitting element sets but not connected to another EM signal emitting element sets; or be electrically connected to any suitable set of EM signal emitting elements. The processor can additionally or alternatively be connected to the communication module, sensor(s), power storage system, base, or any other suitable lighting system component. The processor preferably controls power provision to the EM signal emitting elements and/or communicates information to external devices using the EM signal emitting elements by controlling the pulse rate of the EM signal emitting elements (e.g., by controlling the PWM rate of the LED), but can alternatively control power provision and/or communicate information by controlling the current provided to the EM signal emitting element or controlling any other suitable parameter of the power provided to the EM signal emitting element. The external device can be a remote device (e.g., outside of a communication range for the EM signal, protocol, etc., physically separated from the lighting system 21, by a wall or other EM barrier, outside of a line of sight, etc.), a collocated device (e.g., connected to the lighting system 21, by a wire), or any other suitable device. The processor can additionally function to record lighting system 21, data and send the lighting system 21, data to a device. The processor is preferably a PCB, but can alternatively be any other suitable computing unit.

The processor can additionally include a power conversion module that functions to convert power source power to power suitable for the EM signal emitting element. The power conversion module can be a voltage converter, power conditioning circuit, or any other suitable circuit. The processor can additionally include digital memory that functions to store settings. The settings can be for each EM signal emitting element, each set of EM signal emitting elements, the desired parameters of the cumulative light output, or any other suitable setting. The memory is preferably volatile, but can alternatively, be any other suitable memory.

### 2.3 Substrate.

The substrate of the lighting system 21, functions to mechanically support and mount the EM signal emitting element. The substrate can additionally function to supply power and/or operation instructions to the EM signal emitting elements from the processor or power supply (e.g., the included herein; fragrant warmer 12, or power storage system 800). The substrate is preferably mounted to an end of the housing and is preferably encapsulated between the housing and the cover (e.g., the diffuser). However, the substrate can be arranged in any other suitable position within the lighting system 21. The substrate is preferably a PCB, but can alternatively be any other suitable surface.

The substrate preferably defines a first broad face, and can additionally define a second broad face opposing the first broad face, sides, or define any other suitable surface. The EM signal emitting elements are preferably mounted to a single broad face (e.g., the first broad face), but can alternatively be mounted to the sides, the second broad face, or to any other suitable portion of the substrate. The substrate profile (e.g., cross section) preferably mirrors that of the housing, but can alternatively be different. The substrate profile can be circular, polygonal, irregular, or be any other suitable shape. The substrate can be substantially flat (planar), curved (e.g., concave, (convex bulb 21, as shown in FIG. 9) semi-spherical, etc.), as polygonal (e.g., cylindrical, cuboidal, pyramidal, octagonal, etc.), or have any other suitable configuration. The substrate can be rigid, flexible, or have any other suitable material property.

The substrate is preferably reflective or can additionally include a reflector, such that light directed toward the substrate from the light emitting elements can be reflected away from the substrate. The reflector can be substantially flat, curved, or have any other suitable configuration. The reflector can be textured, smooth, or have any other surface feature. However, the substrate can be matte, dark (e.g., such that the reflected light is absorbed), or have any other suitable property.

### 2.4 Cover and Housing.

the lighting system 21, can additionally include a cover that functions to cooperatively encapsulate the EM signal emitting elements with the substrate. The cover can function to mechanically protect the EM signal emitting elements. The cover can function to change the properties of EM signals emitted by the elements. The cover is preferably arranged proximal the first broad face of the substrate, but can alternatively be otherwise arranged. The cover and substrate (or housing) preferably cooperatively entirely encapsulate the EM signal emitting elements, but can alternatively partially encapsulate the EM signal emitting elements or encapsulate any other suitable portion of the light emitting elements. The cover can be transparent, opaque, translucent, or have any other suitable optical property. The cover can trace the substrate profile or have a different profile. The cover can be cylindrical (e.g., with rounded corners), convex bulb 21, or have any other suitable shape. The cover can be arranged with a broad face substantially perpendicular the active face(s) of the EM signal emitting elements, the broad face of the substrate, or arranged in any other suitable configuration. The cover can be made of plastic, metal, ceramic, or any other suitable material.

The cover can additionally function as a diffuser, or the system can additionally Include a diffuser. The diffuser functions to diffuse and blend the light emitted by the individual EM signal emitting elements or different EM signal emitting Element sets. The diffuser is preferably translucent and diffuses light, but can Alternatively, be a color filter or include any other suitable optical property. The diffuser can additionally include a communication Feature that permits data to be communicated through the diffuser (e.g., using Visible light, invisible light, another EM signal, or any other suitable wireless Communication mechanism). The communication feature can be an aperture Through the diffuser thickness (e.g., a light pipe), a set of apertures or opaque Features (e.g., printed dots) that selectively permit permeation of the Communication wavelength but diffuses EM signals of other wavelengths, or be any Other suitable feature that permits communication therethrough. The Communication feature can be arranged along the entirety of the diffuser side, along a portion of the diffuser side (e.g., portion proximal the housing, portion Distal the housing), along a broad face of the diffuser (e.g., along the flat Surface of the diffuser), along a diffuser edge, extend along the entirety or portion of the diffuser arcuate face, or along any other suitable portion of the diffuser. The Communication feature is preferably substantially aligned with a normal vector of the active surface of the EM signal emitting element communicating the Information (e.g., an IR LED), but can alternatively be at an angle to the normal Vector, or be arranged in any other suitable configuration. The lighting system 21, can additionally include a housing that functions to Encapsulate, protect, and support the lighting system components. The Housing can additionally or alternatively be thermally coupled to and function as a heat sink for the warmer and lighting system 21, components. The housing is Preferably mounted proximal the second broad face of the substrate, but can Alternatively, be mounted to the first broad face or be otherwise arranged. The Housing can be made of metal, ceramic, plastic, or any other suitable material. The housing can additionally include a base that functions as a power Supply. The base can function to physically retain and electrically connect the Lighting system 21, to the LED Fragrant oil warmer 12. The base is the warmer 12, which can be the standard bulb base Configured to connect to the warmer 12, (e.g., an Edison screw base).

The base 8, as shown in phantom in FIG. 9, is preferably mounted to an end of the housing opposing the substrate, but can alternatively be mounted to any other suitable portion of the housing. The base 8, can be electrically connected to the processor, power storage system, EM signal emitting elements, sensors, communication modules, and/or other lighting system components, but can alternatively be electrically connected to any other suitable component.

### 2.5 Sensors.

The lighting system 21, can additionally include a set of sensors that function to measure ambient environment parameters, system parameters, or any other suitable parameter. These measurement values can be used to adjust EM signal emitting element operation (e.g., adjust the intensity of emitted light from the warmer 12, the color temperature of emitted light from the warmer 12, turn the elements on or off from the warmer 12, etc.), change communicated control information, interpret control information, or be used in any other suitable manner.

Sensors can include position sensors (e.g., accelerometer, gyroscope, etc.), location sensors (e.g., GPS, cell tower triangulation sensors, triangulation system, trilateration system, etc.), temperature sensors, pressure sensors, light sensors (e.g., camera, CCD, IR sensor, etc.), current sensors, proximity sensors, clocks, touch sensors, vibration sensors, or any other suitable sensor. The sensors can be connected to and transmit data to the processor and/or communication module.

The lighting system 21, can additionally include a communication module that functions to communicate data to and from the lighting system 21 (e.g., as a transceiver). The communication module preferably includes a receiver, and can additionally include a transmitter. The communication module is preferably a wireless communication module, such as a Zigbee, Z-wave, or Wifi chip, but can alternatively be a short-range communication module, such as Bluetooth, BLE beacon, RF, IR, or any other suitable short-range communication module, a wired communication module, such as Ethernet or powerline communication, or be any other suitable communication module.

The communication module can include an antenna that functions to transmit or receive wireless data. The antenna can extend through the substrate, extend along the housing (e.g., along a longitudinal axis, about the housing perimeter, etc.), extend along the cover, or extend along any other suitable portion of the lighting system 21. The antenna can extend through the thickness of the substrate (e.g., from the second face to the first face), along or parallel a broad face of the substrate, at an angle through the substrate, or through any other suitable portion of the substrate. The antenna can extend through a central portion of the substrate (e.g., coaxially with the central axis offset from the central axis, etc.), through a periphery of the substrate, or along any other suitable portion of the substrate.

The lighting system 21, can include one or more communication modules. In variants including multiple communication modules (e.g., such that the lighting system 21, is a multiradio system), each communication module can be substantially similar (e.g., run the same protocol), or be different. In a specific example, a first communication module can communicate with a remote router, while a second communication module functions as a border router for devices within a predetermined connection distance. The multiple communication modules can operate independently and/or be incapable of communicating with other communication modules of the same lighting system 21 or can operate based on another communication module of the lighting system (e.g., based on the operation state of, information communicated by, or another operation-associated variable of a second communication module). However, the lighting system 21, can include any suitable number of communications modules connected and/or associated in any other suitable manner. The lighting system 21, can additionally or alternatively include a router (e.g., a Wifi router), an extender for one or more communication protocols, a communication protocol translator, or include any other suitable communication module.

### 2.7 Power Storage System

The lighting system 21, can additionally include a power storage system that functions to store power, provide power, and/or receive power. The power storage system can be electrically connected to the processor, power supply (e.g., base), and/or other lighting system components. The power storage system can be arranged within the housing, arranged external the housing, or arranged in any other suitable position. The power storage system can be a battery (e.g., a rechargeable secondary battery, such as a lithium chemistry battery; a primary battery), piezoelectric device, or be any other suitable energy storage, generation, or conversion system.

### 3. Lighting System Examples.

In a first variation, the system 21, includes a first and second set of light emitting elements, wherein both sets are configured to emit visible light. A light parameter (e.g., color temperature, wavelength, to control the warming process inside of the warming device 12, etc.) is preferably fixed for both the first and second sets of light emitting elements. The first and second sets of light emitting elements are preferably configured to emit light having a first and second fixed parameter value, respectively. The first and second sets of light emitting elements cooperatively form a lighting system 21, having a dynamically adjustable parameter, wherein the adjustable parameter is preferably the parameter that is fixed for each set of light emitting elements.

In response to receipt of a target value for the fixed parameter from the device, the processor preferably controls the relative pulse rate, intensity, or other operation parameter of the first and second sets of light emitting elements to meet the target value. However, the processor can control the light emitting elements in any other suitable manner. The parameter value of the subsequently emitted light can additionally be verified using a light sensor on the system or the device, or be verified in any other suitable manner.

In a first example of the first variation, the first set of light emitting elements are configured to emit light having a first color temperature, and the second set of light emitting elements are configured to emit light having a second color temperature. The processor preferably controls the relative power

provision to the first and second sets of light emitting elements such that the resultant color temperature emitted by the entirety of the lighting system meets a target value, wherein the target value can be received from the warming device 12.

In a specific example, the first set of light emitting elements are configured to emit white light having a 6,000K color temperature, and the second set of light emitting elements are configured to emit white light having a 2,700K color temperature. In response to receipt of a target color temperature of 4,000K, the processor can control lighting system 21, operation to provide a first pulsing rate to the first set of light emitting elements and a second pulsing rate to the second set of light emitting elements, wherein the first pulsing rate can be 22% of the second pulsing rate. The pulse rates are preferably determined based on a selected total light intensity, which can also be received from the device. Alternatively, the pulse rate can be determined based on a maximum pulse rate or current as determined by a dimmer switch or any other suitable mechanism. However, the pulse rate can be otherwise determined. The processor can additionally accommodate for differences in the number, characteristics (e.g., quality), or any other parameter of light emitting elements between each set. For example, the processor can provide more than 22% of the second current to the first set of light emitting elements when the first set includes less light emitting elements than the second set.

In a second variation, the system includes a first set of light emitting elements configured to emit visible light and a second set of light emitting elements configured to emit light at a wavelength outside of the visible spectrum. The processor preferably controls operation of the first and second sets of light emitting elements independently, in response to independent operation instructions received from the device. More specifically, the processor can supply power to the first set of light emitting elements in response to receipt of a target operation parameter for the first set of light emitting elements, and supply power to the second set of light emitting elements in response to receipt of a target operation parameter for the second set of light emitting elements. In a specific example, the system can include a first set of light emitting elements configured to emit white light and a second set of light emitting elements configured to emit infrared light. The system can additionally include a third set of light emitting elements configured to emit white light at a second color temperature, wherein the first set of light emitting elements are configured to emit white light at a first color temperature and the processor can selectively control the first and second sets of light emitting elements to achieve a target parameter value. However, the system can include any other suitable sets of light emitting elements.

In response to receipt of a white light operation command, the processor can provide power to the first set of light emitting elements. In response to receipt of an infrared operation command, the processor can provide power to the second set of light emitting elements. Alternatively, the first and/or second sets of light emitting elements can be automatically controlled, based on stored user settings (e.g., stored on-board or remotely), historical use of the set by a user, historical use of the set by a population, or controlled in any other suitable manner. The infrared light can function to provide better IR coverage for IR applications, such as security applications (e.g., for security camera illumination), monitoring applications (e.g., baby monitoring), night imaging applications, plant growth applications, data transfer applications, or any other suitable application, which can result in higher resolution images. The infrared light is preferably used with a secondary system that includes an infrared sensor, but the system can alternatively include an infrared sensor. In the latter variation, a first lighting system 21, can detect the light emitted by a second lighting system.

In one variation of infrared-containing light bulb use, the infrared-containing light bulb is used to provide the infrared light for a security system. The light bulb is preferably distributed about a monitored space, wherein the light bulbs are preferably installed into the light fixtures of the monitored space. The infrared lights are preferably powered in response to shutoff or a decrease in power provision to the set of visible-light emitting elements, but can alternatively be powered on in response to the instantaneous time meeting a predetermined time (e.g., turned on at 6:00 PM), powered on in response to the ambient light falling below a predetermined threshold, or powered in response to any other suitable event.

In a specific example, a subset of the infrared-containing light bulbs in the monitored space are initially powered. The light bulbs forming the powered subset is preferably substantially evenly distributed about the space, but can alternately be the light bulbs located over a space entry (e.g., window, door, etc.), or be any other suitable subset of light bulbs. Alternatively, or additionally, a subset of the infrared elements on each powered light bulb can be powered, while the remaining infrared elements can remain off. Alternatively, or additionally, the powered subset can be powered with a low current or pulsed at a low rate, such that the infrared elements provide low-intensity infrared light. The set of powered lighting system 21, preferably cooperatively illuminate the entire space, but can alternatively illuminate a subset of the space through the exterior 20, of the warmer 12.

In response to motion detection by a sensor, the remaining infrared elements of a light bulbs in the space can be powered, wherein the current provided to or pulse rate of the infrared elements is preferably high, but can alternatively be low or have any other suitable magnitude. Alternatively, or additionally, the first set of visible-light emitting elements can be powered in response to motion detection. An image of the room can additionally be recorded prior to turning the first set of lights on. The image can additionally be processed to determine whether the detected moving object is recognized, wherein the lighting system 21, is preferably operated in a first mode (e.g., a nightlight mode) in response to a recognized object and operated in a second mode (e.g., a full power mode) in response to a non-recognized object. In the nightlight mode, current having a predetermined magnitude or power having a predetermined pulse rate can be supplied to the visible lights of all or a subset of lighting systems. In one example, current can be supplied to the lighting systems proximal the moving object, wherein the location of the moving object can be determined based on the infrared light and sensor measurement analysis.

In another example, the infrared light emitted by the lighting system 21, can function to create a thermal map of a monitored space, wherein the thermal map can be used to adjust operation of an HVAC system (e.g., air conditioning system). Alternatively, a temperature control system can control the lighting system 21, to emit infrared light in response to the temperature falling below a temperature threshold.

The infrared light can be used to communicate information from lighting system 21, to a peripheral device. The peripheral device is preferably within a line of sight of the lighting system 21, independent of visible-light emitting element operation, but can alternatively be arranged in any other suitable location. The information can be communicated by pulsing or otherwise adjusting the intensity, saturation, or any other suitable light parameter of the emitted infrared light. The information can additionally or alternatively, be communicated by changing which infrared light emitting element is emitting the infrared light, or communicated in any other suitable manner. The information can be data generated by the lighting system 21, data received by the lighting system 21, from a remote or connected device, or be any other suitable information. The information can be received by a peripheral device, such as a television, mobile phone, or any other suitable device, and converted into a control signal or any other suitable device information for the peripheral device. Examples of control signals that can include operation instructions, media (e.g., audio/video transmission), device identification, device connection information, or any other suitable information. Different infrared light emitting elements of the same lighting system can simultaneously communicate information to two different peripheral devices, but can only communicate information to a single peripheral device, a predetermined set of peripheral devices, or any other suitable number of peripheral devices. The communicated information can be the same piece of information or be different pieces of information, wherein the different pieces of information can be simultaneously communicated by different infrared light emitting elements of the same lighting system 21, or by different lighting systems. The lighting system 21, can additionally function to receive data communicated by the peripheral device. The information can be communicated through a data channel (e.g., Wifi), EM signals emitted by the peripheral device (e.g., modulated IR light), or communicated in any other suitable manner. In a third example, the light emitted by the light emitting elements (e.g., IR, visible light, a combination thereof, etc.) can be used to repel insects, arachnids, or other pests. This example can include determining the location of a user (e.g., using a secondary sensor, the location of a user device associated with the user, etc.) and directing the infrared light or other EM signal to repel pests away from the user location or any other suitable location (e.g., location of food). Directing the infrared light or other EM signal to repel pests away from the user location can include illuminating the area surrounding the user location with IR light, directing EM signals that attract insects at an area distal the user, or otherwise drawing insects away from the user location.

### 4. Method

The method of lighting system 21, operation include: receiving control instructions at a lighting system 21, and controlling a set of EM signal emitting elements based on the control instructions. The method can enable the lighting system 21, to selectively emit light having a range of lighting parameters, even though the lighting system 21, only includes light emitting elements having fixed lighting parameters. The method can additionally enable the lighting system 21, to double as a remote-control extender for the fragrant oil warmer system 12.

The method is preferably performed with the system described above. More preferably, the method is performed with the fragrant warmer device 12, wherein the lighting systems 21, and devices are preferably associated with a common user account. However, the method can be performed with any other suitable system. Receiving the control instruction at the lighting system 21, functions to provide instructions for lighting system 21, operation. The control instruction can be received at the lighting system 21, by the communication module, but can alternatively be received in any other suitable manner by any other suitable component. The control instruction is preferably received from a sending device, wherein the sending device sends the control instruction or a derogatory instruction to the lighting system 21, but can alternatively be received from any other suitable source. The instructions can be sent directly, through a secondary lighting system 21, through a communication network (e.g., Wifi), through a remote computing system, or through any other suitable communication channel. The instructions can be sent using the communication protocol in which the control instruction was received, a second communication protocol, or any other suitable protocol. The sending device can be a user device 60 (e.g., wherein the control instruction is entered by a user on a user interface, received by the user device at an input device, etc.), a second lighting system, a remote computing system (e.g., remote server system), an external device (e.g., connected outlet, accessory, computing system, etc.), or any other suitable source. The sending device can receive the control instruction (or a precursor thereof) from a user (and therefore be the receiving device), receive the control instruction from a second sending device, automatically generate the control instruction (e.g., based on instantaneous and historical sensor measurements, etc.), or otherwise determine the control instructions. The sending device can additionally process the control instruction, such as by compressing the information, associating the control instruction with an endpoint (e.g., fragrant warmer 12, identifier, lighting system 21, identifier, EM signal emitting element, etc.), transforming the control instruction (e.g., into the modulation pattern or operation instructions), associating the control information with contextual information (e.g., sensor measurement values recorded within a threshold time period of control instruction receipt, timestamps, etc.), associating the control information with user account information (e.g., a user account identifier), associating the control information with any other suitable information, or otherwise processing the control information.

The sending device is preferably associated with the same user account as the lighting system 21 but can alternatively be associated with a different user account. The control instruction can be automatically generated, manually entered (e.g., user-generated), or otherwise generated by the sending device. The control instruction can include one or more lighting instructions (e.g., target EM signal emission parameter values), fragrant warmer 12, instructions (e.g., for appliance control), context parameter values (e.g., timestamps, weather information, sensor measurements, etc.), endpoint identifiers (e.g., a unique address for the lighting system 21, an appliance identifier, etc.), or include any other suitable information. The method can additionally or alternatively determine the type of control instruction. For example, the method can include determining whether the control instruction is an appliance instruction or a lighting instruction, wherein the type of control instruction can be determined based on the length of the control instruction, the communication protocol of the control instruction, an endpoint address included within the control instruction, the commands within the control instruction, or be determined in any other suitable manner. A first set of EM signal emitting elements (e.g., visual light emitting elements) are preferably controlled when the control instructions include lighting instructions (e.g., according to the mixing variant below), and a second set of EM signal emitting elements (e.g., invisible light emitting elements, IR light emitting elements, etc.) are preferably controlled when the control instructions include appliance instructions (e.g., according to the external device control variant below). However, the EM signal emitting elements of one or more lighting systems can be otherwise controlled. The control instructions can include instructions for a single endpoint (e.g., a single fragrant warmer device 12, a single lighting system 21, etc.), instructions for multiple endpoints (e.g., for the fragrant warmer device 12, multiple lighting systems, etc.), or instructions for any suitable set of endpoints

The control instruction can additionally include trigger events associated with the information, wherein the information is used when the trigger event is met. For example, the control instruction can include a trigger event, including a set of sensor measurement values, associated with the lighting instructions, wherein the lighting instructions are performed when the lighting system sensors record measurements substantially matching the set of sensor measurement values. The control information can additionally include associations between different pieces of the control information. For example, a lighting instruction can be associated with the fragrant warmer device 12, instruction, wherein the lighting instruction and fragrant warmer device 12, instruction is to be concurrently performed. However, the control instruction can include any other suitable information.

The method can additionally include determining secondary control instructions based on the control instruction. The secondary control instructions can be for the fragrant oil warmer device 12, or other devices (e.g., lighting systems adjacent the appliance when the control instruction is an appliance instruction; appliance instructions when the control instruction is a lighting instruction, etc.), for the target device, or for any other suitable device. The secondary control instructions can be determined (e.g., generated, selected, calculated, etc.) based on the control instruction and instantaneous contextual parameter values, based on the control instruction alone, or be determined based on any other suitable information. In one example, the control instruction can be an appliance instruction for the thermostat to lower the temperature, while the secondary control instructions can be to concurrently lower the color temperature inside of the warmer of visible light emitted by the lighting systems proximal the user (e.g., proximal the user device, such as a smart phone). Alternatively, or additionally, when the user historically increases the color temperature of the emitted visible light when the room temperature is lowered, the secondary control instruction can be to concurrently increase the color temperature of the emitted visible light inside of the warmer 12. In a second example, the control instruction can be an appliance instruction for the television to change the channel, wherein the secondary control instructions can be to adjust the color temperature and/or hue of the emitted visible light based on the dominant color palette of the resultant channel inside of the warmer 12. However, the secondary control instruction can be otherwise determined.

Individually controlling a set of EM signal emitting elements based on the control instructions function to concurrently emit EM signals having one or more properties from the lighting system. Independent EM signal emitting element set operation is preferably controlled by the processor, but can alternatively be controlled by any other suitable control system.

In a first variation, individually controlling the elements includes operating a first set of light emitting elements at a first intensity and operating a second set of light emitting elements at a second intensity, wherein the light emitting elements cooperatively emit visible light having a target light parameter value. In this variation, the first set of light emitting elements includes different light emitting elements from the second set of light emitting elements, and the first intensity is different from the second intensity.

In a second variation, individually controlling the elements includes concurrently operating a set of visible light emitting elements according to a lighting instruction, and operating a set of communication EM signal emitting elements (communication elements) according to an appliance instruction. The set of visible light emitting elements can be operated according to a lighting instruction as discussed in the first variation. The set of communication elements can be operated according to the device instruction by determining a modulation pattern corresponding to the device instruction (e.g., that will communicate the device instruction to the warmer 12), and modulating the waveform of the power supplied to the communication elements according to the modulation pattern. Operating the set of communication elements can additionally or alternatively include selecting the communication element most proximal the appliance, and controlling only the selected communication element according to the modulation pattern. However, the element sets can be otherwise individually controlled.

The method can additionally include learning control instructions based on contextual patterns, which functions to automatically determine and control the device and lighting systems according to user preferences. The user preferences can be individual user preferences, global user preferences, or user preferences for any other suitable set of users. The user preferences can be stored in association with the user account, stored by the user device, stored by the lighting systems, or be stored in any other suitable manner. The control instructions and associated contextual patterns are preferably learned by the remote computing system, but can alternatively be learned by the user device, one or more lighting systems, or by any other suitable computing system. The control instructions are preferably learned from historical control instructions and them associated contextual parameter values, but can alternatively be received from a user, or otherwise determined.

In one variation, learning control instructions based on contextual patterns includes: receiving the control instruction; determining context parameter values associated with control instruction receipt; assigning the context parameter values with the control instructions to form a control record; and extracting a context parameter value pattern associated with the control instruction from a plurality of control records. The lighting system 21, is preferably automatically controlled according to the control instruction in response to the occurrence of an instantaneous set of context parameter values substantially matching the context parameter value pattern. However, the control instructions and associated contextual pattern can be otherwise determined.

The context parameter values are preferably values measured within a predetermined time threshold of control instruction receipt (e.g., concurrent with control instruction receipt, within 10 seconds of receipt, etc.), but can alternatively be recorded at any other suitable time. The context parameter values can be a timestamp; a weather variable value (e.g., received from a remote server system); the device operation state; a lighting system operation state; a lighting plurality operation state; a sensor measurement value (e.g., ambient noise, temperature, light, etc.) from one or more lighting systems, connected outlets, connected switches, or other connected systems; a pattern or combination of device operation states; or be a value of any other suitable parameter indicative of context. Automatic system control based on satisfaction of the contextual parameters can include: automatically generating and/or communicating the warmer's instructions to the appliances via the lighting systems; automatically generating and/or communicating lighting instructions to the lighting systems; or automatically controlling any other suitable device. The control instructions can be generated and/or communicated by a control system, wherein the control system can be the remote computing system (e.g., server system), a user device, a lighting system or set thereof, or by any other suitable set of computing systems. The control system can receive sensor measurements, control instructions, or any other suitable information from the connected devices (e.g., lighting systems, user devices, connected outlets, etc.) at a predetermined frequency, as the measurements are recorded, or at any other suitable time.

In one example, the method can include operating a first fragrant oil warmer 12, according to a first set of control instructions in response to a first contextual pattern being met, and operating a second set of appliances according to a second set of control instructions in response to a second contextual pattern being met. The first fragrant oil warmer 12, can be the same or different. The first and second set of control instructions can be the same or different.

In a specific example, the method can include: automatically turning on the first fragrant oil warmer 12, when a user enters the house, and automatically shutting off the second fragrant warmer/or both when a user leaves the house or goes to sleep. In this specific example, the first contextual pattern can be the user entering the house (e.g., determined based on the geographic location of the user device, proximity to beacons, based on power provision to one or more lighting systems within the house); and the second contextual pattern can be the user turning off the fragrant oil warmer 12, (e.g., power provision cessation).

In response to determination of user entry, the method can include: concurrently communicating a first set of control instructions associated with the first context parameter pattern to a plurality of appliances through a plurality of lighting systems (e.g., to turn on the fragrant oil warmer that the user usually turns on). The method can additionally include storing power in power storage devices on-board each of the plurality of lighting systems in response to power receipt at lighting system 21.

In response to cessation of power provision to the lighting system 21, the method can include concurrently communicating a second set of control instructions to the plurality of warming device through the plurality of lighting systems (e.g., to turn off the device that the user usually turns off). Because no more power is being supplied to the lighting systems at this time, each lighting system 21, can use the power stored by the respective power storage devices (e.g., batteries) to: determine that power provision has ceased; send a power cessation notification to the control system; receive control instructions from the control system, and send the control instructions to the fragrant oil warmer. However, the system can be otherwise controlled based on contextual patterns.

### 4.1 Mixing.

In a first variation this method includes: receiving a target EM signal emission parameter value at the lighting system 21, and individually controlling different sets of EM signal emitting elements to emit an EM signal having parameter values substantially matching the target EM signal emission parameter value. In this variation, receiving the control instruction includes: receiving a target EM signal emission parameter value at the lighting system 21; and individually controlling a set of EM signal emitting elements based on the control instructions include: individually controlling different sets of EM signal emitting elements to emit an EM signal having parameter values substantially matching the target EM signal emission parameter value. This method variant functions to provide a lighting system 21, made from lighting elements having static lighting properties, with dynamically adjustable lighting capabilities of the fragrant warming device 12.

In one example, the method includes: receiving a target light parameter value (e.g., color temperature value), determining the relative intensities for a first and second light emitting element set to meet the target light parameter value, and operating the first and second light emitting element sets at the respective intensities to cooperatively emit light having substantially the target light parameter value. In a first specific example, the lighting system 21, has a first plurality of light emitting elements and a second plurality of light emitting elements. The first plurality of light emitting elements emit white light having a fixed, cool color temperature, but still have enough heat to emit the fragrance from the container 23, (e.g., without the capability to emit light having another color temperature). The second plurality of light emitting elements emits white light having a fixed, warm color temperature. The target color temperature is between the cool and warm color temperatures. The method determines how bright the first plurality of light emitting elements should be operated, and how bright the second plurality of light emitting elements should be operated, such that the light emitted by the lighting system 21, (i.e., the light cooperatively emitted by the first and second pluralities of light emitting elements and blended by the diffuser) has a color temperature substantially matching the target color temperature. In a second specific example, the first plurality of light emitting elements emits light having a first fixed hue (e.g., red) and the second plurality of light emitting elements light having a second fixed hue (e.g., red), each plurality without capability to emit light having another hue. In response to receipt of a control instructions specifying a target hue of purple, the first and second plurality of light emitting elements can be controlled to both emit the same intensity of light. The intensity of each plurality can substantially match that specified by the control instructions, be half that specified by the control instructions, or be any other suitable intensity. In response to receipt of a control instruction specifying a target hue of red, the first plurality of light emitting elements can be operated at the specified intensity, while the second plurality of light emitting elements can be operated at a low intensity or shut off. However, the first and second pluralities can be otherwise operated to achieve a target parameter value.

Receiving a target EM signal emission parameter value at the lighting system 21, functions to provide the lighting system 21, with control instructions for EM signal emitting element operation. The target EM signal emission parameter value (target parameter value) is preferably received as part of a set of control instructions (as discussed above), but can alternatively be otherwise received. The EM signals emission parameter value can be a specific wavelength (e.g., hue, color temperature, saturation, etc.), intensity, direction, phase, or be any other suitable parameter value for the warmer 12.

Individually controlling different sets of EM signal emitting elements functions to control the lighting system 21, to emit an EM signal having parameter values that substantially match the target EM signal emission parameter value. Individually controlling different sets of EM signal emitting elements can include: determining the relative operation parameters for multiple sets of EM signal emitting elements, based on the target parameter value and the respective emission properties of the sets; and controlling each set according to the respective operation parameter.

The operation parameters that can be determined include the operation intensity (e.g., the amplitude or emission intensity for each set), the percentage of each set to be operated (e.g., in variants wherein individual subsets can be independently controlled), or include any other suitable operation parameter. The operation parameters can calculate, empirically determined (e.g., by dynamically adjusting the relative operation parameters and measuring the emitted light with an external sensor), selected from a graph or chart, or otherwise determined.

Determining the relative operation parameters can include calculating an operation parameter ratio for the multiple sets, based on the respective fixed operation parameter for each set and the target operation parameter value. For example, if the first and second sets have a 1,700K and 10,500K color temperature, respectively, and the target color temperature is 5,00K, then the operation ratio for the first set can be 62.5% more than the second set. The first set can be operated at an intensity that is 62.5% higher than the intensity of the second set, have 62.5% more elements in operation compared to the second set, or be controlled based on the calculated ratio in any other suitable manner and all set temperature can generate enough heat to volatile the fragrance inside of the reservoir 23, located inside of the warmer 12.

Determining the relative operation parameters can additionally include accounting for a second target operation parameter value. For example, the control instruction can specify both a target color parameter (e.g., color temperature, hue, saturation) and a target intensity for the cooperatively emitted light, wherein the method can scale the respective intensities of each light emitting element set based on the target intensity (e.g., to substantially meet the target intensity). The second target operation parameter value can be accounted for by scaling the determined intensities, applying the determined ratio to the second target operation parameter value, using the second target operation parameter value as the maximum value for any light emitting element set, or be otherwise accounted for.

Determining the relative operation parameters can additionally include accommodating for differences in perceived intensities of the first and second sets. For example, when a first light having a warm color temperature (e.g., 1,700K) and a second light having a cold color temperature (e.g., 10, 5000K) are emitted at the same intensity, the first light can be perceived as less intense by a user, wherein the method can accommodate for this discrepancy by increasing the intensity of the first light. Accommodating for the differences can include weighting the respective fixed operation parameter value for the set when determining the ratio, correcting the ratio by a correction factor, or otherwise accommodating for the difference in perception. However, the relative operation parameters can be otherwise determined.

Controlling each set according to the respective operation parameter preferably includes determining a pulse width modulation pattern (PWM pattern) corresponding to the relative operation parameter for the set and providing power to the light emitting element according to the PWM pattern (e.g., as described above). However, each set can be otherwise controlled based on the operation parameter.

### 4.2 External Device Control.

The method includes: receiving the fragrant oil warmer 12, instruction for another warmer, identifying a lighting system 21, proximal the fragrant oil warmer 12, determining a modulation pattern to communicate the control instruction to the fragrant oil warmer 12, and controlling an EM signal emitting element of the lighting system 21, according to the modulation pattern. In this variation, receiving the control instruction at the lighting system 21, includes: receiving the fragrant oil warmer 12, instruction or derivatory instructions, and individually controlling a set of EM signal emitting elements based on the control instructions includes: controlling an EM signal emitting element of the lighting system 21, according to the modulation pattern.

This method functions to extend the communication range of a remote control. The method can additionally function to target communication to the fragrant oil warmer 12, such that other appliance adjacent the target appliance (e.g., within the same room as the target appliance) do not receive the control instruction and/or are not controlled by the control instruction. This can be useful when multiple appliances of similar type are closely arranged (e.g., when multiple televisions are closely arranged), but only one appliance is to be controlled. The method can additionally function to simultaneously send communications to multiple appliances, whether adjacent (e.g., in the same room) or remote (e.g., in different rooms, buildings, or other geographic locations). The method can additionally function to translate control instructions between communication protocols, which can expand the number of remote-control devices that can be used to control the appliance. The second method variation or any portion thereof can be performed in conjunction with, concurrently with, or independently from first method variation performance. However, the system can be used in any suitable manner and/or perform any other suitable functionality.

Receiving the fragrant oil warmer 12, instruction functions to provide the fragrant oil warmer 12, instruction to the system for subsequent processing and/or transmission. The fragrant oil warmer 12, instruction can be received by a user device, the lighting system, 21, a secondary lighting system, a remote computing system, or by any other suitable system. The receiving system is preferably associated with the same user identifier (e.g., user account, Wifi network, IP address, etc.) that the lighting system 21, (and/or appliance) is associated with, but can alternatively be unassociated with any user identifier, associated with a different user identifier, or otherwise related to the lighting system 21. The fragrant oil warmer 12, instruction can be received from a sending device 80 (e.g., in the manner discussed above), received from the user (e.g., at a user input device, at a graphical interface, etc.), but can alternatively be received from any other suitable source.

The fragrant oil warmer 12, instruction is preferably a set of instructions meant for the fragrant oil warmer 12, and can include control instructions (e.g., on/off instructions, setting selection, setting control, etc.), display information (e.g., AN information), or include any other suitable information. The fragrant oil warmer 12, is preferably a home appliance (e.g., device designed for domestic or household functions.

In one variation, the method can additionally include sending data indicative of the fragrant oil warmer 12, instruction to the lighting system 21. The data indicative of the fragrant oil warmer 12, instruction can be the device instruction, as received by the receiving device; be a derivatory instruction, determined (e.g., computed, translated, selected, etc.) based on the device instruction (e.g., the modulation pattern); or be any other suitable data associated with the device instruction. The data indicative of the device instruction is preferably sent by the device receiving the device instruction (receiving device) via a wireless communication method, but can alternatively, be sent by any other suitable computing system in any other suitable manner.

The receiving device is preferably external the lighting system 21 but can be arranged in any other suitable position. The receiving device can be proximal the lighting system 21, (e.g., within communication range for a short-range communication protocol, within communication range for a lighting system-hosted local network, within the same room as the lighting system, within a predetermined distance of the lighting system, etc.), remote from the lighting system (e.g., outside of the communication range for a short-range communication protocol, located in a different room or building from the lighting system, outside a predetermined distance of the lighting system, etc.), or be arranged in any suitable physical position relative to the lighting system 21.

The data indicative of the appliance instruction can be sent before the modulation pattern is determined (e.g., wherein the lighting system 21, determines the modulation pattern), after the modulation pattern is determined (e.g., wherein the data is the modulation pattern or a precursor thereof), or at any other suitable time. The data indicative of the device instruction is preferably sent after the lighting system 21, proximal the appliance is identified but can alternatively be sent at any other suitable time.

In a first example, receiving the device instructions can include: receiving the fragrant oil warmer 12, instructions at a device from a user; and sending the device instructions to a first lighting system from the device in response to appliance instruction receipt. The method can additionally include forwarding the fragrant oil warmer 12, instructions (or derivatory instructions) to a second lighting system, remote server system, or any other suitable endpoint. In a second example, receiving the appliance instructions can include: receiving the fragrant oil warmer 12, instructions at a device from a user; sending the appliance instructions to a remote server system from the device in response to appliance instruction receipt; and sending the appliance instructions (or derivatory instructions) to the lighting system 21, from the remote server system. In a third example, receiving the fragrant oil warmer 12, instructions can include: generating the device instructions at a remote server system, user device, or lighting system; and sending the device instructions (or derivatory instructions) to the lighting system 21. However, the fragrant oil warmer 12, instructions can be otherwise received and/or generated.

Identifying a lighting system 21, proximal the fragrant oil warmer 12, functions to identify the lighting system 21, with the highest probability of communicating the device instruction to the device, such that the device instruction or derivatory instruction can be sent only to the identified lighting systems This can function to reduce data traffic and reduce unintentional appliance control.

In a first variation, the lighting system(s) proximal the fragrant oil warmer 12, (e.g., local the device) can be uniquely identified, wherein the control instruction (or derivatory information) can be addressed to the lighting system 21, and sent to the lighting system 21. The addressed lighting system 21, can be sent through a common communication channel shared by all connected devices (e.g., associated with the user account), wherein the lighting system 21, identified by the address selectively receives the information (e.g., pulls the information), and the other lighting systems ignore the information. Alternatively, or additionally, the control instructions can be sent only to the targeted lighting system by selectively connecting to a local network hosted by the lighting system based on the address, and communicating the control instruction through the local network. Alternatively, or additionally, the information can be sent peer to peer (e.g., verified through a digital handshake). Alternatively, or additionally, the information can be sent in a targeted direction (e.g., broadcast in a physical direction, such as in the direction of a room in which the lighting system is located). However, the information can be otherwise targeted at the lighting system 21.

In a second variation, the lighting system 21, can remain unidentified, and the fragrant oil warmer 12, instructions can be broadcast to all lighting systems associated with the user account, a lighting system within a predetermined physical range, all lighting systems connected to a common wireless network, or to any other suitable set of lighting systems. The fragrant warming lighting system 12, can be identified by the receiving device, by an intermediary device (e.g., a remote server system), or by any other suitable device. The lighting system is preferably identified by a lighting system 21, identifier, but can alternatively be otherwise identified. The lighting system 21, identifier can be globally unique, unique within the population of lighting systems associated with the user account, unique within the population of lighting systems within a geographic area or connected to a common wireless network, generic/shared, or be otherwise related to other lighting system identifiers. The lighting system identifier can be automatically determined (e.g., assigned by the manufacturer, automatically assigned upon user setup based on other lighting systems already associated with the user account or the communication network 70, etc.), manually determined (e.g., assigned by a user), or be otherwise determined.

The identified lighting system 21, is preferably associated with the fragrant oil warmer 12, identifier but can alternatively be any other suitable lighting system. In one variation, the method includes identifying the appliance identifier based on the control instructions, and identifying the lighting system 21, based on the appliance identifier. The device identifier can be definitively determined or probabilistically determined (e.g., wherein the target device is the one that has the highest probability of being the target, based on context, etc.). The device identifier is preferably associated with the user account but can alternatively be unassociated with the user account. The device identifier can be determined based on control instruction parameters (e.g., control instruction length, communication protocol, etc.); based on the content of the control instructions, wherein the instructions are compared against a database of instructions; based on an endpoint identifier included in the control instructions; or otherwise determined. In one example, a television identifier is identified in response to the control instructions being below a predetermined size or length, while an air conditioning unit identifier is identified in response to the control instructions being above a second size or length. However, the target appliance identifier can be otherwise determined.

In a first variation, identifying the lighting system 21, proximal the warmer 12, can include: retrieving a lighting system 21, identifier associated with the fragrant oil warmer 12, from a database. In a second variation, identifying the lighting system 21, proximal the appliance can additionally or alternatively include: sequentially sending and controlling the lighting system 21, to emit the fragrant oil warmer 12, instruction (or derivatory instruction) to different lighting systems associated with the user account until the device receives the device instruction, which can be determined based on a detected change in device operation (e.g., wherein a lighting system 21, sensor or other sensor on another system, such as an outlet or light switch, records a measurement indicative of the change). However, the lighting system 21, proximal of the fragrant oil warmer 12, can be otherwise identified.

The method can additionally include associating the lighting system 21, with the fragrant oil warmer 12, which can be subsequently used in the first variation of identifying the lighting system 21, proximal the device. Associating the lighting system with the fragrant oil warmer 12, can include: determining an association between the lighting system 21, and the device, and storing the lighting system 21, identifier in association with the device. The lighting system(s) within communication range of the fragrant oil warmer 12, (e.g., local lighting systems, proximal lighting systems, etc.) are preferably associated with the fragrant oil warmer 12, but any other suitable lighting system can be associated with the device.

Storing the lighting system 21, identifier in association with the fragrant oil warmer 12, can include; storing the identifier for the lighting system 21, (lighting system identifier) in association with the identifier for the device (appliance identifier) in a remote computing system or other storage system; storing the fragrant oil warmer 12, identifier in the lighting system 21, memory; or otherwise associating the lighting system 21, with the warmer 12. One or more lighting systems can be associated with each device, and one or more devices can be associated with each lighting system. The association between the lighting system 21, and the fragrant oil warmer 12, can be determined: manually (e.g., received from a user, wherein the user enters or selects the lighting system 21, identifier and the appliance identifier); pseudo-automatically; automatically; or otherwise determined. In one variation of pseudo-automatic association determination, the user device is placed or held next to the fragrant oil warmer 12, (e.g., in front of, adjacent the appliance sensor, between the appliance and the lighting system 21, etc.). Individual lighting systems are then independently operated at different times (e.g., controlled by a user device, remote computing system, etc.). The user device (Or user) notifies the system when light (visible or invisible) emitted by the lighting system is proximal or illuminates the fragrant oil warmer 12, and/or the light sensor of the user device. The identifiers of the lighting system(s) in operation when the fragrant oil warmer 12, was illuminated are then associated with the device. However, the association can be otherwise pseudo-automatically determined.

In one variation of automatic association determination, the system can determine the relative position between a lighting system 21, and an outlet, wherein the outlet is electrically connected to the warmer 12, and identifies or is otherwise associated with the fragrant oil warmer 12. The position of the lighting system relative to the outlet can be automatically determined (e.g., based on trilateration using signals emitted and detected by the lighting system 21, and/or outlet, determined from an image of the room, etc.), received from a user, or otherwise determined. The fragrant oil warmer 12, connected to the outlet can be: manually identified; automatically identified based on data transfer from the device to the outlet; automatically identified based on the amount of power drawn, pattern of drawn power; or otherwise identified. The device can be assumed to have a rear face facing the outlet, with the sensing face distal the outlet, but can be assumed to be in any other suitable position. The lighting system 21, having light emitting elements directed toward the outlet is preferably associated with the fragrant oil warmer 12, but any other suitable lighting system can be associated with the fragrant oil warmer 12. However, the lighting system 21, can be otherwise associated with the outlet.

Associating the lighting system 21, with the appliance can additionally or alternatively include associating one or more specific EM signal emitting element(s) of the lighting system 21, with the fragrant oil warmer 12, wherein the EM signal emitting element identifier(s) are preferably subsequently identified, and elements operated to communicate the control instruction to the warmer 12. In this variation, the EM signal emitting elements of the lighting system 21, can be individually indexed and controlled. In operation, when a control instruction is to be communicated to the fragrant oil warmer 12, the specific EM signal emitting element communicates the control instruction to the fragrant oil warmer 12, while the other EM signal emitting elements of the lighting system 21, can operate in a different mode (e.g., in a dim, off, or standby mode). This functions to target communication to the target fragrant oil warmer 12, which can limit inadvertent control instruction communication to other appliances. This also functions to allow a single lighting system to concurrently control (or communicate control instructions to) multiple appliances.

As above, associating one or more EM signal emitting elements with the fragrant oil warmer 12, can include: determining an association between the EM signal emitting element(s) with the warmer 12, and storing identifier(s) for the EM signal emitting element(s) with the warmer identifier. However, the EM signal emitting elements can be otherwise associated with the warmer. The association can be stored in a similar manner to lighting system association storage, or be stored differently. The association can be stored by the lighting system 21, by a second lighting system, by a remote server, by a user device, or by any other suitable system.

The EM signal emitting element associated with the fragrant oil warmer 12, is preferably a communication signal emitting element (e.g., an invisible signal emitting element, such as an IR light emitting element, RF emitting element, visible light emitting element, etc.), but can alternatively be a visible light emitting element or be any another suitable EM signal emitting element. The EM signal emitting element(s) within communication range of the warmer 12, (e.g., local lighting systems, proximal lighting systems, etc.) are preferably associated with the fragrant oil warmer 12, but any other suitable EM signal emitting element can be associated with the fragrant oil warmer 12.

The association between the EM signal emitting element and the fragrant oil warmer 12, can be determined: manually (e.g., received from a user, wherein the user enters or selects the EM signal emitting element identifier and the device identifier); pseudo-automatically; automatically; or otherwise determined. However, the EM signal emitting element can be dynamically associated with the device (e.g., the control instruction is communicated by different EM signal emitting elements until the device operates according to the control instructions) or be otherwise associated with the warmer 12.

In one variation of association determination, the user device is placed or held next to the fragrant oil warmer 12, (e.g., in front of, adjacent the appliance sensor, between the device and the lighting system, etc.). Individual EM signal emitting element sets of the lighting system 21, are sequentially operated (e.g., scrolled through) to project light from different EM signal emitting elements (e.g., different elements arranged in different arcuate or radial positions; projecting light radially outward, etc.) automatically, in response to an arcuate manual input, or operated in any other suitable manner. The EM signal emitting element sets preferably have fixed, known angular, radial, or other position relative to the lighting system 21.

The user device notifies the system when an EM signal emitted by the EM signal emitting element is proximal or illuminates the warmer 12, and/or the light sensor of the user device. The identifiers of the EM signal emitting element(s) in operation when the fragrant oil warmer 12, and/or user device light sensor was illuminated are then associated with the fragrant oil warmer 12.

In one example of a variant, individual visible light emitting element sets of the lighting system 21, are sequentially operated (e.g., scrolled through) to project light from different visible light emitting elements (e.g., different elements arranged in different arcuate or radial positions) automatically, in response to an arcuate manual input, or controlled in any other suitable manner. The light emitting element sets preferably have fixed, known angular, radial, or other position relative to the lighting system. The user device (or user) notifies the system when a visible light emitted by the visible light emitting element is proximal or illuminates the fragrant oil warmer device. The identifiers of the visible light emitting element(s) in operation when the fragrant oil warmer 12, was illuminated are then identified, and the EM signal emitting element(s) associated with the visible light emitting element(s) that were in operation are then associated with the device. The EM signal emitting element associated with the warmer is preferably the EM signal emitting element proximal the identified visible light emitting element (e.g., arcuately or radially adjacent the identified visible light emitting element, within the same group as the identified visible light emitting element, etc.), wherein the position of the EM signal emitting element relative to the identified visible light emitting element on the lighting system is known, but can alternatively be an EM signal emitting element configured to direct light in substantially the same direction as the identified visible light emitting element, or be any other suitable EM signal emitting element. In a specific example, associating the EM signal emitting element (e.g., invisible light emitting element, infrared light emitting element, etc.) with the appliance includes: scrolling through a set of visual light emitting elements having predetermined angular positions on the lighting system 21, including, at each of a set of timestamps, concurrently operating a visual light emitting element in a high mode and operating a remainder of the set in a low mode; storing each of the set of timestamps with an identifier for the visual light emitting element concurrently operated in the high mode; receiving an association notification from a user device, the association notification including an association timestamp and an identifier for the device; determining a reference timestamp from the set of timestamps substantially matching the association timestamp; determining the visual light emitting element identifier, stored in association with the reference timestamp, as a reference visual light emitting element identifier; determining an identifier for an invisible light emitting element located adjacent the visual light emitting element identified by the reference visual light emitting element identifier; and storing the invisible light emitting element identifier in association with the warmer identifier. However, the invisible light emitting element can be otherwise associated with the warmer. In a second variation of association determination, the EM signal emitting element proximal the warmer or appliance (e.g., most proximal the warmer) can be determined after the lighting system associated with the warmer is automatically identified (e.g., using the methods described above). In this variation, the lighting system can determine its rotational orientation relative to the, such that a lighting system reference point position relative to the warmer is known; retrieve a known position of the EM signal emitting elements relative to the reference point; and determine the EM signal emitting element(s) most proximal the warmer and/or the EM signal emitting element(s) configured to direct EM signals toward the warmer based on the lighting system rotational position and EM signal emitting element positions relative to the lighting system 21, reference point.

In a first variation, determining the position of the lighting system 21, reference point relative to an external reference point includes determining the orientation of the lighting system 21, using an onboard compass or other positioning system. In a second variation, determining the position of the lighting system reference point relative to an external reference point includes selectively powering a single or subset of EM signal emitting elements (indexing light emitting elements) and detecting the light on a mobile device including a light sensor (e.g., a camera or other light sensor). The location of the mobile device can be recorded in response to a detected light parameter (e.g., intensity) surpassing a predetermined threshold. Because the emission direction of the EM signal emitting element is known and the location of the indexing EM signal emitting element relative to the remainder of EM signal emitting elements on the lighting system is known, the orientation of the indexing EM signal emitting element and remainder EM signal emitting elements can be determined once the recipient device geographic location is recorded. However, the position of one or more EM signal emitting elements relative to the appliance can be otherwise determined and associated with the device. Determining a modulation pattern to communicate the control instruction to the device functions to process the fragrant oil warmer instruction into instructions for EM signal emitting element operation. The modulation pattern (e.g., PWM modulation pattern) can be determined by the receiving device (e.g., the device initially receiving the control instruction), by the sending device (e.g., the device sending the control instruction), the lighting system operating its EM signal emitting elements according to the modulation pattern, or by any other suitable device. The modulation pattern is preferably the pattern required to communicate an equivalent of the control instructions (or derivative thereof) using the EM signal emitting element(s), but can alternatively be any other suitable modulation pattern. Alternatively, the method can include generating operation instructions for a lighting system emitter (e.g., RF operation instructions, A/V instructions, etc.) to communicate the control instructions to the fragrant oil warmer. However, the control instruction (or derivatory instruction) can be otherwise communicated to the fragrant oil warmer 12, via the lighting system 21.

Determining a modulation pattern can additionally include selecting the communication protocol, which functions to translate control instructions in a first communication protocol to control instructions in a second communication protocol. The communication protocol is preferably selected based on the communication protocol(s) accepted by the device (wherein the accepted communication protocols can be retrieved from a database or otherwise determined), but can alternatively be otherwise determined. Different EM signal emitting element types can be used when different communication protocols are selected, wherein the operated EM signal emitting element preferably corresponds to the selected communication protocol. Alternatively, different modulation patterns can be selected based on the selected communication protocol. However, the selected communication protocol can be otherwise used. Controlling an EM signal emitting element of a lighting system according to the modulation pattern functions to communicate the control instruction (or derivatory instruction) to the appliance. Alternatively, this can include controlling one or more emitters of the lighting system (e.g., RF emitters, microwave emitters, BLE transceivers, etc.) according to the operation instructions, which were determined based on the control instructions. However, the control instruction can be otherwise communicated to the fragrant oil warmer 12.

The EM signal emitting element controlled according to the modulation pattern can be: all EM signal emitting elements of the lighting system 21; the EM signal emitting element associated with the device (e.g., as determined above); or be any other suitable EM signal emitting element or set thereof. In one example, controlling the EM signal emitting element according to the modulation pattern includes: operating the identified infrared light emitting element according to the modulation pattern; and operating a second infrared light emitting element of the plurality according to a second modulation pattern different from the modulation pattern. The EM signals emitting element is preferably controlled by the processor of the lighting system according to the modulation pattern (e.g., by regulating power provision to the element), but can alternatively be controlled by the remote computing system (e.g., remote server system), a user device, or be controlled by any other suitable system.

Again referring to FIG. 9, warmer 12 is illustrated using a resistance heater rod as warming device 14. Here, resistance heater rod 14 can be secured to or formed as part of base 22. Also, warmer 12 can, in some embodiments, further include a lighting device 21 such as, but not limited to a light emitting diode (LED). Cover 12 shields substantially all of resistance heater rod 14 and, when present, lighting device 21. As discussed in detail above, cover 12 can include decorative pattern 36 defined on an external surface 12 thereof and/or the cover can be at least partially translucent. Resistance heater rod 14, and when present resistance heater rod device 14, can be selectively placed in electrical communication with a power source (not shown) such as, but not limited to, a battery or an electrical outlet. Fan 7 and speaker 4 can be selectively placed in electrical communication with a power source (not shown) such as, but not limited to, battery or an electrical outlet. Wherein said speaker 4 produce audio sound through speaker grille 11 located on the exterior of the cover 12, coupled to an interior side of the speaker 4, and configured to output stereo sound through a room area. Cover 12 shields substantially all of speaker system 4, fan 7, and when present, lighting device 21.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

What is claimed is:

1. A warmer for scented oil comprising:
an enclosure having a base and a crown cover removably positioned on said top rim, said cover having an opening defined at an upper apex of said cover; an open channel container reservoir formed in said cover at said upper apex around an outer periphery of said opening, said open channel container reservoir being configured to receive the scented oil therein; and
a warming device in heat conductive relationship with said enclosure so that scented oil in said open channel container reservoir is warmed by desired said warming devices, and
a warming device in electrically conductive relationship with said enclosure so that smart bulb in said open channel is illuminate light colors through the exterior of the warmer, and
a warning device in electrically conductive relationship with said enclosure so that speaker in said base is in connection with a sensor/computer device, configured to control one or more of a fan speed and a lighting level based upon instructions received from an internet connection
wherein said enclosure encloses substantially all said fan, speaker, and warming device.

2. The warmer of claim 1, wherein said warming device comprises a lighting device having an LED smart light bulb/interconnected resistance heater rod, fan, and speaker with air vents for extra-ventilation for smart bulb.

3. The warmer of claim 2, wherein said lighting device, heater rod, fan and speaker is in said enclosure, so that said enclosure shields substantially all said LED smart bulb, fan, speaker, resistance heater rod.

4. The warmer of claim 3, further comprising a decorative pattern defined on an external surface of said enclosure that is at least partially translucent.

5. The warmer of claim 3, further comprising a lighting device in said enclosure so that said enclosure shields substantially all said lighting devices, fan, speaker.

6. The warmer of claim 2, wherein lighting device comprises a light emitting diode.

7. The warmer of claim 1, wherein said warming device comprises a LED smart bulb removably position able on said base so that said cover shields substantially all said LED smart bulb with said resistance heating rod and fan being at a center of said opening.

8. The warmer of claim 1, further comprising at least one vent opening defined between said cover and said base.

9. The warmer of claim 3, wherein warming device comprising a audio speaker coupled to an interior side of the grille, and configured to output stereo sound through a second area of the plurality of openings; and.

10. The warmer of claim 1, further comprising a scented oil or wax in a semi-solid form or a solid form pre-filled in said open channel container reservoir.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)
